(19) United States
(12) Patent Application Publication
(10) Pub. No.: US 2003/0203833 A1
(43) Pub. Date: Oct. 30, 2003

(54) Association of asthma with polymorphisms in the cysteinyl leukotriene 2 receptor

(76) Inventors: Diane Michele Ignar, Durham, NC (US); Sreekumar Pillai, Durham, NC (US)

Correspondence Address: DAVID J LEVY, CORPORATE INTELLECTUAL PROPERTY, GLAXOSMITHKLINE, FIVE MOORE DR., PO BOX 13398, RESEARCH TRIANGLE PARK, NC, 27709-3398, US

(21) Appl. No.: 10/131,332

(22) Filed: Apr. 24, 2002

(51) Int. Cl. A61K 31/00; C12Q 1/68

(52) U.S. Cl. 514/1; 435/6

(57) ABSTRACT

Polymorphisms in the cysteinyl-leukotriene receptor 2 gene and their association with asthma are described.

### FIELD OF THE INVENTION

The present invention relates to polymorphisms in the cysteinyl-leukotriene receptor 2 gene and asthma.

### BACKGROUND OF THE INVENTION

Asthma is one of the most frequent chronic diseases with a prevalence of up to 10% (Jarvis and Burney, Br. Med. J. 316:607 (1988)). Both genetic and environmental factors contribute to the overall phenotype. Family studies have shown an increased frequency of asthma in first-degree relatives (Marsh, Allergol. Immunopathol. (Madr.) Suppl 9:60 (1981), Townley et al, J. Allergy Clin. Immunol. 77:101 (1986)). Environmental exposure to allergens, pollutants, and viral respiratory infections are important in the development of asthma (Boushey et al, Am. Rev. Respir. Dis. 121:389 (1980), Cookson and Moffat, Hum. Mol. Genet. 9:2359 (2000)). The interaction between the genetic and environmental factors in the pathogenesis of asthma is not fully understood. Asthma is the most common chronic childhood disease in developed nations and carries substantial direct and indirect economic cost (Lenny, Pediatr. Pulmonol. Suppl. 15:13 (1997)). The prevalence of asthma and other allergic diseases has risen over the past 2 decades (McNally, et al, Soc. Sci. Med. 46:729 (1998)) and the cost of treating the disease in United States is approximately US $6 billion per annum (Smith et al, Am. J. Respir. Crit. Care Med. 156:787 (1997)).

In view of the impact of asthma, better methods of identifying children at risk, and of assessing the efficacy of anti-asthma therapeis, would be beneficial.

### SUMMARY OF THE INVENTION

A first aspect of the present invention is a method of identifying a subject at increased risk of asthma, by detecting the allelic forms of the A601G polymorphism in the Cysteinyl leukotriene 2 receptor gene. A person homozygous for the A allele is at increased risk for asthma compared to a subject with at least one G allele.

### BRIEF DESCRIPTION OF THE FIGURES

FIG. 1 provides the sequence of the CysLT2 receptor gene (SEQ ID NO:1), with the start codon underlined (264-266) and stop codon underlined (1302-1304). The polymorphic sites are indicated by bold underlined text: coding SNP A601 G (864), noncoding SNP TSC3P1 (A/G at 2796) and TSC3P2 (A/G at 2960).

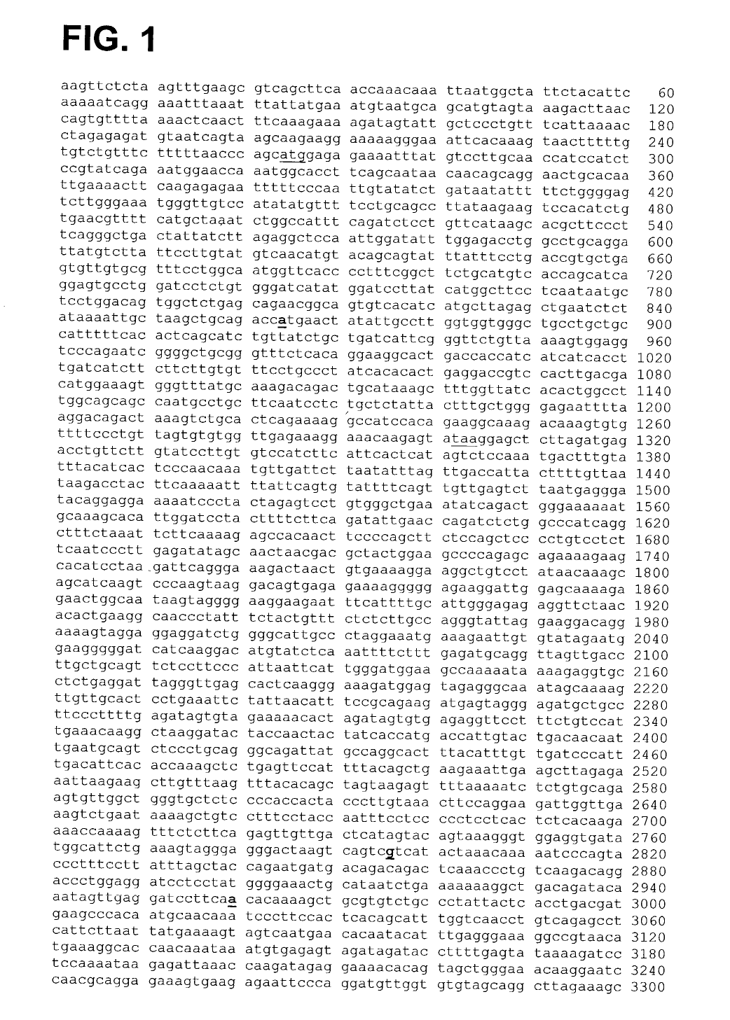

FIG. 2 provides the amino acid sequence (SEQ ID NO:2) encoded by the above nucleotide sequence, with the polymorphic amino acid (met/val) shown in bold underlined text.

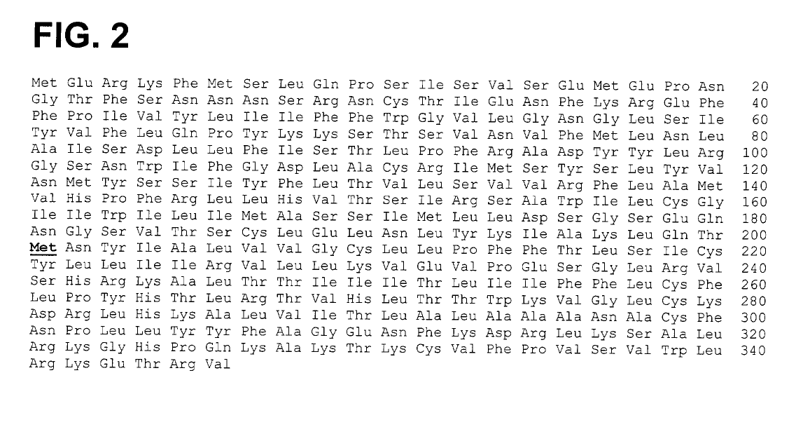

### DETAILED DESCRIPTION

Leukotrienes are a family of eicosinoids which form part of a much larger group of compounds synthesized from arachadonic acid. The cysteinyl leukotrienes (CysLTs), LTC4, LTD4, and LTE4, previously known as slow reacting substance of anaphylaxis, or SRS-A, are derived from arachidonic acid via oxygenation and dehydration by 5-lipoxygenase followed by specific glutathione addition by LTC4 synthase.

The CysLTs mediate their biological actions through two pharmacologically defined G-protein-coupled receptors (GPCRs), named the CysLT1 and CysLT2 receptors. (See WO 01/59105 (Glaxo Group Limited); Nicosia, Monaldi Arch. Chest Dis. 54:242 (1999); Takasaki et al., Biochem. Biophys Res. Commun. 274:316 (2000); Heise et al., J. Biol. Chem. 275:30531 (2000); Nothacker et al., Mol. Pharmacol. 58:1601 (2000); Nicosia et al., Pulm. Pharmacol. Ther. 14:3 (2001)). LTB₄ is produced mainly by macrophages and neutrophils and stimulates neutrophil chemotaxis, enhances neutrophil-endothelial cell interactions and stimulates neutrophil activation leading to degranulation and the release of mediators, enzymes and superoxides. The cysteinyl leukotriene receptors respond to LTD₄, LTE₄, LTC₄ and LTF₄, however the occurrence in vivo of LTF₄ is unclear.

Cysteinyl leukotrienes contract airway smooth muscle, increase microvascular permeability, stimulate mucus secretion, decrease mucociliary clearance and recruit eosinophils into the airways. The CysLT2 receptor has been documented pharmacologically to be expressed in guinea pig trachea and ileum, ferret trachea and spleen, sheep bronchus, and human pulmonary and saphenous vein preparations. At the CysLT2 receptor subtype, the agonist potency rank order is LTC4=LTD4>>LTE4 and LTE4 is a partial agonist.CysLT1 receptor-specific leukotriene receptor antagonists, such as montelukast, zafirlukast and pranlukast are currently used to control bronchoconstriction and inflammation in asthmatic patients. CysLT1 is mainly detected in lung smooth muscle cells, macrophages, spleen and peripheral blood lymphocytes and has not been detected in heart.

As used herein, a “genetic subset” of a population consists of those members of the population having a particular genotype. In the case of a biallelic polymorphism, a population can potentially be divided into three subsets: homozygous for allele 1 (1,1), heterozygous (1,2), and homozygous for allele 2 (2,2). A ‘population’ of subjects may be defined using various criteria, e.g., individuals being treated with a certain therapeutic or diagnosed with a certain medical condition, individuals of a defined ethnicity or demographic group, etc.

As used herein, a subject that is “predisposed to” or “at increased risk of” a particular phenotypic response based on genotyping will be more likely to display that phenotype than an individual with a different genotype at the target polymorphic locus (or loci).

“Genetic testing” (also called genetic screening) as used herein refers to the testing of a biological sample from a subject to determine the subject's genotype; and may be utilized to determine if the subject's genotype comprises alleles that either cause, or increase susceptibility to, a particular phenotype (or that are in linkage disequilibrium with allele(s) causing or increasing susceptibility to that phenotype).

“Linkage disequilibrium” refers to the tendency of specific alleles at different genomic locations to occur together more frequently than would be expected by chance. Alleles at given loci are in complete equilibrium if the frequency of any particular set of alleles (or haplotype) is the product of their individual population frequencies A commonly used measure of linkage disequilibrium is r:

$$\begin{matrix}r = \frac{{\hat{Δ}}_{AB}}{\sqrt{( {\tilde{π}}_{A} + {\hat{D}}_{A} ) ( {\tilde{π}}_{B} + {\hat{D}}_{B} )}} \\ where \\ {\tilde{π}}_{A} = {\tilde{p}}_{A} ( 1 - {\tilde{p}}_{A} ) , {\tilde{π}}_{B} = {\tilde{p}}_{B} ( 1 - {\tilde{p}}_{B} ) , {\hat{D}}_{A} = {\tilde{P}}_{AA} - {\tilde{p}}_{A}^{2} , {\hat{D}}_{B} = {\tilde{P}}_{BB} - {\tilde{p}}_{B}^{2} \\ {\hat{Δ}}_{AB} = \frac{1}{n} {n}_{AB} - 2 {\tilde{p}}_{A} {\tilde{p}}_{B}\end{matrix}$$

nr² has an approximate chi square distribution with 1 degree freedom for biallelic markers. Loci exhibiting an r such that nr² is greater than 3.84, corresponding to a significant chi-squared statistic at the 0.05 level, are considered to be in linkage disequilibrium (BS Weir 1996 Genetic Data Analysis II Sinauer Associates, Sunderland, Md.).

Alternatively, a normalized measure of linkage disequilibrium can be defined as:

$${D}_{AB}^{′} = \{ \begin{matrix}\frac{{D}_{AB}}{min ( {p}_{A} {p}_{B} , {p}_{a} {p}_{b} )} , & {D}_{AB} < 0 \\ \frac{{D}_{AB}}{min ( {p}_{A} {p}_{b} , {p}_{a} {p}_{B} )} , & {D}_{AB} > 0\end{matrix}$$

The value of the D′ has a range of −1.0 to 1.0. When statistically significant absolute D value for two markers is not less than 0.3 they are considered to be in linkage disequilibrium.

The present studies investigated the presence of three polymorphisms in the CysLT2 receptor gene, and the occurrence of asthma.

Sample Composition and Clinical Evaluation

Asthma families were studied at 2 collection centers: (1) the Department of Medicine, University of Minnesota Medical School, Minneapolis, Minn. (Min); and (2) Department of Respiratory Medicine, Hvidovre University Hospital, DK-2650 Hvidovre, Denmark (Den). In the selection of these families, at least two siblings with clinical asthma were required. The proband had the following criteria for the diagnosis of asthma: (1) at least 2 out of the 3 categories including of cough, wheezing, and dyspnoea should be recurrent and (2) a documentation of an increase in FEV by 15% of predicted value, minimum 300 ml) after a bronchodilator or (b) a positive methacholine inhalation challenge (PC₂₀ FEV₁<10 mg/ml). The following exclusion criteria were followed:

1. Birth weight less than 4.4 pounds;

2. Systematic vasculitis involving the lungs;

3. Congenital or acquired pulmonary diseases at birth;

4. Uncorrected congenital heart disease;

5. Severe cardiac disease;

6. Current medications that interfered with phenotypes that could not be stopped, such as beta blocking agents;

7. Isolated occupation induced asthma.

All subjects were evaluated by using standard protocols. Baseline spirometry was performed by according to American Thoracic Society (ATS) criteria. Skin Prick Tests (SPT) for the common allergens (mites, animal, insects, pollen and mould) were conducted.

The Denmark collection was composed of 268 families, 985 samples with genotypes, 367 children with Physician's diagnosis of asthma (PDA), 120 with Bronchial hyper-reactivity (BHR) and 275 with atopic asthma. The Minnesota collection constituted 83 Caucasian families, 317 samples with genotypes, 168 children with PDA, 144 with strict asthma, 122 with BHR and 139 with atopic asthma.

In addition to the family samples, a matched Caucasian case control collection from North Carolina was also evaluated (one hundred cases and 100 controls, “asthma-1” collection). This collection is from Duke University Medical Center, Durham, N.C. The primary diagnostic criteria for this collection was a definite physician's diagnosis for the presence of asthma (PDA) or not asthma. Skin prick tests to common allergens and standard spirometric measurements were also evaluated in this collection.

Phenotypes:

The following phenotypes were evaluated. 1) PDA; 2) strict asthma, 2 or more classic symptoms (cough, wheeze and shortness of breath) and a positive methacholine challenge test or bronchodilator reversibility; 3) bronchial hyper-reactivity (BHR), positive methacholine response at or below 10 mg/ml of methacholine and 4) atopic asthma, physician's diagnosis and positive results on at least one skin allergen tests.

Genotyping:

DNA was isolated from whole blood or lymphoblastoid cell lines. The Single Nucleotide Polymorphisms (SNP) genotypes were generated using the 5′ nuclease assay with TaqMan® (Applied Biosystems, Foster City, Calif.) fluorogenic probes and the products were read on an ABI PRISM® 7700 Sequence Detection System (Applied Biosystems).

Statistical Analysis:

The association of the asthma related phenotypes with the markers are tested by transmission disequilibrium test comparing the frequencies of the alleles transmitted to affected children to those not transmitted using Transmit (Dudbridge et al, Am. J. Hum. Genet. 66:2009 (2000)), for the family data. Association analysis for the case control population was done using Fisher's exact test (Zaykin et al, Genetica 96:169 (1995)). Haplotype analysis of the family data was done using Transmit (Dudbridge et al, 2000) and by a method using EM algorithm for the case control data (Zaykin et al, “Testing association of statistically inferred haplotypes with discrete and continuous traits in samples of unrelated individuals, Human Heredity (in press)).

Hardy-Weinberg Equilibrium (HWE) Analysis:

The departure from HWE is tested using a Chi square test, by testing the difference between the expected (calculated from the allele frequencies) and observed genotype frequencies.

Linkage Disequilibrium (LD) Analysis:

The LD between two markers is given by D_{AB}=p_{AB}−p_{A}p_{B}, where p_{A} is the allele frequency of A allele of the first marker, p_{B} is the allele frequency of _{B} allele of the second marker, and p_{AB} is the joint frequency of alleles A and B on the same haplotype. A commonly used measure of LD can be calculated as follows.

Where

$$\begin{matrix}{\hat{Δ}}_{AB} = \frac{1}{n} {n}_{AB} - 2 {\tilde{p}}_{A} {\tilde{p}}_{B} \\ r = \frac{{\hat{Δ}}_{AB}}{\sqrt{( {\tilde{π}}_{A} + {\hat{D}}_{A} ) ( {\tilde{π}}_{B} + {\hat{D}}_{B} )}}\end{matrix}$$

nr² has an approximate chi square distribution with 1 df.

According to the present methods, a compound may be screened for variation in its effectiveness in treating asthma among genetic subpopulations of subjects. the phenotypic response of subjects to an asthma treatment may include the magnitude, duration, or occurrence of a positive response to treatment, or the magnitude, duration or occurrence of an unwanted side effect, or the absence of any response. Methods of correlating genotype with phenotypic response to treatment include administering the therapeutic to a population of subjects, obtaining biological samples from the subjects, genotyping polymorphic allelic sites as identified herein, and correlating the genotype of the subjects with their phenotypic response (e.g., response to therapeutic treatment).

Polymorphic alleles may be detected by determining the DNA polynucleotide sequence, or by detecting the corresponding sequence in RNA transcripts from the polymorphic gene, or where the nucleic acid polymorphism results in a change in an encoded protein by detecting such amino acid sequence changes in encoded proteins; using any suitable technique as is known in the art. Polynucleotides utilized for typing are typically genomic DNA, or a polynucleotide fragment derived from a genomic polynucleotide sequence, such as in a library made using genomic material from the individual (e.g. a cDNA library). The polymorphism may be detected in a method that comprises contacting a polynucleotide or protein sample from an individual with a specific binding agent for the polymorphism and determining whether the agent binds to the polynucleotide or protein, where the binding indicates that the polymorphism is present. The binding agent may also bind to flanking nucleotides and amino acids on one or both sides of the polymorphism, for example at least 2, 5, 10, 15 or more flanking nucleotide or amino acids in total or on each side. In the case where the presence of the polymorphism is being determined in a polynucleotide it may be detected in the double stranded form, but is typically detected in the single stranded form.

As is well known genetics, nucleotide and amino acid sequences obtained from different sources for the same gene may vary both in the numbering scheme and in the precise sequence. Such differences may be due to inherent sequence variability within the gene and/or to sequencing errors. Accordingly, reference herein to a particular polymorphic site by number (e.g., CysLT2R A601G) will be understood by those of skill in the art to include those polymorphic sites that correspond in sequence and location within the gene, even where different numbering/nomenclature schemes are used to describe them.

### EXAMPLE 1

### Primers and Probes

Marker Name: CysLT2R 3P1 (A>G)

Allele 1=A

Allele 2=G

Probes:
allele 1 =

(SEQ ID NO:3)

6FAM-ACTAAGTCAGTCATCATACTAAACAAAAATCC-TAMRA
allele 2 =

(SEQ ID NO:4)

VIC-ACTAAGTCAGTCGTCATACTAAACAAAAATC-TAMRA
Primers:

(SEQ ID NO:5)

CysLT2R 3P1 A>G F GGGTGGAGGTGATATGGCATT

(SEQ ID NO:6)

CysLT2R3P1 A>G R TCTGTCTGTCATCATTCTGGTAGCT

All probes and primers were supplied at 100 uM in water.

Marker Name: CysLT2R 3P2 (A>G)

Allele 1=A

Allele 2=G

Probes:
allele 1 =

(SEQ ID NO:7)

6FAM-TTGAGGATCCTTCAACACAAAAGCTGC-TAMRA
allele 2 =

(SEQ ID NO:8)

VIC-TTGAGGATCCTTCA GCACAAAAGCTG-TAMRA
Primers:

(SEQ ID NO:9)

CysLT2R 3P2 A>G F AGACAGACTCAAACCCTGTCAAGAC

(SEQ ID NO:10)

CysLT2R3P2 A>G R GCTTCATCGTCAGGTGAGTAATAGG

All probes and primers were supplied at 100 uM in water.

Marker Name: CysLT2R A601G (CysLT2R Met201Val)

Allele 1=A

Allele 2=G

Probes:
allele 1 =

(SEQ ID NO:11)

6FAM-TGCTAAGCTGCAGACCATGAACTATATTGC-TAMRA
allele 2 =

(SEQ ID NO:12)

VIC-CTAAGCTGCAGACCGTGAACTATATTGCC-TAMRA
Primers:

(SEQ ID NO:13)

CysLT2R A601G A>G F CACATCATGCTTAGAGCTGAATCTC

(SEQ ID NO:14)

CysLT2R A601G A>G R CAGATAACAGATGCTGAGTGTGAAAA

All probes and primers were supplied at 100 uM in water.

### EXAMPLE 2

### Results

The above three markers were genotyped in the Denmark, Minnesota and asthma-1 collections, in the Cyslt2R gene. One coding polymorphism (A601G), and 2 noncoding SNPS (TSC3P1 and TSC3P2) that are 3′ to the coding polymorphism were genotyped.

Populations Studied:

(1) Minnesota: 91 families

(2) Denmark: 268 families

(3) Asthma-1: 200 cases and 100 controls (Phenotype PDA)

Den-Min: Combined analysis from Denmark and Minnesota.

The frequencies of different alleles are provided in Table 1 and the results of the association study are provided in Table 2. The allele frequency of CYSLT2R_A601G in Parents with and without asthma in the Denmark and Minnesota populations is shown in Table 3; in the Asthma-1 population, in Table 4. Table 5 shows linkage disequilibrium estimates.

Results and Discussion

The transmission dis-equilibrium test showed significantly lower transmission of the G allele of the marker A601G to the asthmatic children. This association was statistically significant in the combined Denmark and Minnesota families for physician's diagnosis of asthma (PDA), atopic asthma, and strict definition of asthma. The association was statistically significant in the Denmark families for PDA and marginally significant for atopic asthma. The frequency of the G allele was lower in asthmatics compared to non-asthmatics in the asthma-1 case control collection. The affected and un-affected parents (for PDA) from the Denmark and Minnesota collections were examined, and it was found that the frequency of the G allele was lower in affected parents compared to unaffected parents. The markers A601G and CYSLT2R-3P1 are in significant linkage dis-equilibrium (see Table 5) and the haplotype involving the allele G of A601G and allele A of CYSLT2R-3P1 shows significantly lower transmission to the affected children in the combined Minnesota and Denmark families for PDA (p=0.0032).

The A601G single nucleotide polymorphism was found to be associated with Asthma and related phenotypes, and does change the amino acid sequence of CysLt2R from Methionine to Valine at amino acid position 201. The G allele of this polymorphism was found to be associated with resistance to asthma. This polymorphism has been associated with decreased expression of the CysLT2R protein, and also the decreased affinity for leukotrienes to the receptor. This association suggests that compounds that block the CysLT2R receptor are useful for the treatment of asthma.

TABLE-1
Frequency of the Alleles in Different Populations

| Marker | Allele | Minnesota | Denmark | DenMin | Asthma-1 | Pooled |
|---|---|---|---|---|---|---|
| CYSLT2R_3P1 | A | 0.56 | 0.53 | 0.54 | 0.56 | 0.55 |
| CYSLT2R_3P1 | G | 0.44 | 0 47 | 0.46 | 0.44 | 0.45 |
| CYSLT2R_3P2 | A | 0.93 | 0 94 | 0.94 | 0.93 | 0.94 |
| CYSLT2R_3P2 | G | 0.07 | 0.06 | 0.06 | 0.07 | 0.06 |
| CYSLT2R_A601G | A | 0.97 | 0.97 | 0.97 | 0.98 | 0.97 |
| CYSLT2R_A601G | G | 0.03 | 0.03 | 0.03 | 0.02 | 0.03 |

TABLE-2
Results of the Association Study

| | Phenotype | | | | Population |
|---|---|---|---|---|---|
| Marker | ASTHMA | ATP_ASTH | BHR | PDA | Minnesota |
| CYSLT2R_3P1 | 0.315 | 0.307 | 0.874 | 0.676 | Minnesota |
| CYSLT2R_3P2 | 0.295 | 0.520 | 0.822 | 0.528 | Minnesota |
| CYSLT2R_A601G | 0.092 | 0.435 | 0.472 | 0.138 | Minnesota |
| CYSLT2R_3P1 | 0.817 | 0.777 | 0.416 | 0.846 | Denmark |
| CYSLT2R_3P2 | 0.610 | 0.392 | 0.743 | 0.132 | Denmark |
| CYSLT2R_A601G | 0.179 | 0.057 | 0.100 | 0.009 | Denmark |
| CYSLT2R_3P1 | 0.535 | 0.567 | 0.576 | 0.901 | Den-Min |
| CYSLT2R_3P2 | 0.651 | 0.875 | 0.961 | 0.522 | Den-Min |
| CYSLT2R_A601G | 0.037 | 0.042 | 0.102 | 0.002 | Den-Min |
| CYSLT2R_3P1 | | | | 0.77 | Asthma-1 |
| CYSLT2R_3P2 | | | | 0.12 | Asthma-1 |
| CYSLT2R_A601G | | | | 0.31 | Asthma-1 |

(P values from TDT for Denmark and Minnesota and case control test for Asthma-1)

ASTHMA: Strict asthma

ATP_ASTH; Atopic asthma

BHR: Bronchial hyper-reactivity

PDA: Physician's diagnosis of asthma

TABLE 3
Allele Frequency of CYSLT2R_A601G in Parents with Asthma or
without Asthma (Denmark and Minnesota)

| Asthma (PDA) | Number | A/G | G/G | Frequency of G allele |
|---|---|---|---|---|
| NO | 457 | 30 | 0 | 3.28 |
| YES | 142 | 5 | 1 | 2.46 |

TABLE 4
Allele frequency of CYSLT2R_A601G in individuals with asthma or
without asthma (Asthma-1)

| Asthma (PDA) | Number | A/G | G/G | Frequency of G allele |
|---|---|---|---|---|
| NO | 100 | 5 | 0 | 2.50 |
| YES | 200 | 5 | 0 | 1.25 |

TABLE 5
Linkage Disequilibrium Estimates

| | Marker1 | Marker2 | Chi Squared | p Value (Chi Sq.) | LD Correlation R |
|---|---|---|---|---|---|
| Minnesota | CYSLT2R_3P1 | CYSLT2R_3P2 | 14.281 | 0.000157443 | 0.304523 |
| | CYSLT2R_3P1 | CYSLT2R_A601G | 11.0162 | 0.000903191 | 0.266594 |
| | CYSLT2R_3P2 | CYSLT2R_A601G | 1.63404 | 0.201141 | 0.102019 |
| Denmark | CYSLT2R_3P1 | CYSLT2R_3P2 | 41.0963 | 1.45E−10 | 0.297928 |
| | CYSLT2R_3P1 | CYSLT2R_A601G | 4.09937 | 0.0428993 | 0.0938927 |
| | CYSLT2R_3P2 | CYSLT2R_A601G | 3.63906 | 0.0564388 | 0.087899 |
| DenMin | CYSLT2R_3P1 | CYSLT2R_3P2 | 55.067 | 1.16E−13 | 0.298747 |
| | CYSLT2R_3P1 | CYSLT2R_A601G | 12.288 | 0.000455885 | 0.140781 |
| | CYSLT2R_3P2 | CYSLT2R_A601G | 5.24756 | 0.0219776 | 0.0914111 |
| Asthma-1 | CYSLT2R_3P1 | CYSLT2R_3P2 | 35.643 | 2.37E−09 | 0.348782 |
| | CYSLT2R_3P1 | CYSLT2R_A601G | 4.69565 | 0.0302391 | 0.126811 |
| | CYSLT2R_3P2 | CYSLT2R_A601G | 0.275975 | 0.599352 | 0.0303808 |
| Pooled | CYSLT2R_3P1 | CYSLT2R_3P2 | 89.9028 | 2.50E−21 | 0.314316 |
| | CYSLT2R_3P1 | CYSLT2R_A601G | 16.4589 | 4.97E−05 | 0.134339 |
| | CYSLT2R_3P2 | CYSLT2R_A601G | 3.22997 | 0.0723019 | 0.0590282 |

That which is claimed is:

1. A method of identifying a subject at increased risk of asthma, comprising detecting the allelic forms of the A601 G polymorphism in the Cysteinyl leukotriene 2 receptor gene, where a person homozygous for the A allele is at increased risk for asthma compared to a subject with at least one G allele.

2. A method according to claim 1 where the subject is a member of a family having at least one member diagnosed with asthma.

3. A method of stratifying a population receiving pharmaceutical treatment for asthma to detect differences in phenotypic response to the pharmaceutical treatment, by:
(a) genotyping a population of subjects in need of pharmaceutical treatment for asthma, to determine each subject's allelic forms of the A601G polymorphism in the Cysteinyl leukotriene 2 receptor gene;
(b) administering to said subjects a pharmaceutical treatment for asthma;
(c) correlating each subject's phenotypic response to treatment with the subject's genotype, to detect phenotypic responses that are associated with said allelic forms.


SEQUENCE LISTING (14 sequences)

SEQ ID NO: 1 - DNA, 3300 bp, Homo sapiens
  CDS (264)..(1304)
aagttctctaagtttgaagcgtcagcttcaaccaaacaaattaatggctattctacattcaaaaatcaggaaatttaaatttattatgaaatgtaatgcagcatgtagtaaagacttaaccagtgttttaaaactcaactttcaaagaaaagatagtattgctccctgtttcattaaaacctagagagatgtaatcagtaagcaagaaggaaaaagggaaattcacaaagtaactttttgtgtctgtttctttttaacccagcatggagagaaaatttatgtccttgcaaccatccatctccgtatcagaaatggaaccaaatggcaccttcagcaataacaacagcaggaactgcacaattgaaaacttcaagagagaatttttcccaattgtatatctgataatatttttctggggagtcttgggaaatgggttgtccatatatgttttcctgcagccttataagaagtccacatctgtgaacgttttcatgctaaatctggccatttcagatctcctgttcataagcacgcttcccttcagggctgactattatcttagaggctccaattggatatttggagacctggcctgcaggattatgtcttattccttgtatgtcaacatgtacagcagtatttatttcctgaccgtgctgagtgttgtgcgtttcctggcaatggttcacccctttcggcttctgcatgtcaccagcatcaggagtgcctggatcctctgtgggatcatatggatccttatcatggcttcctcaataatgctcctggacagtggctctgagcagaacggcagtgtcacatcatgcttagagctgaatctctataaaattgctaagctgcagaccatgaactatattgccttggtggtgggctgcctgctgccatttttcacactcagcatctgttatctgctgatcattcgggttctgttaaaagtggaggtcccagaatcggggctgcgggtttctcacaggaaggcactgaccaccatcatcatcaccttgatcatcttcttcttgtgtttcctgccctatcacacactgaggaccgtccacttgacgacatggaaagtgggtttatgcaaagacagactgcataaagctttggttatcacactggccttggcagcagccaatgcctgcttcaatcctctgctctattactttgctggggagaattttaaggacagactaaagtctgcactcagaaaaggccatccacagaaggcaaagacaaagtgtgttttccctgttagtgtgtggttgagaaaggaaacaagagtataaggagctcttagatgagacctgttcttgtatccttgtgtccatcttcattcactcatagtctccaaatgactttgtatttacatcactcccaacaaatgttgattcttaatatttagttgaccattacttttgttaataagacctacttcaaaaattttattcagtgtattttcagttgttgagtcttaatgagggatacaggaggaaaaatccctactagagtcctgtgggctgaaatatcagactgggaaaaaatgcaaagcacattggatcctacttttcttcagatattgaaccagatctctggcccatcaggctttctaaattcttcaaaagagccacaacttccccagcttctccagctcccctgtcctcttcaatcccttgagatatagcaactaacgacgctactggaagccccagagcagaaaagaagcacatcctaagattcagggaaagactaactgtgaaaaggaaggctgtcctataacaaagcagcatcaagtcccaagtaaggacagtgagagaaaagggggagaaggattggagcaaaagagaactggcaataagtaggggaaggaagaatttcattttgcattgggagagaggttctaacacactgaaggcaaccctatttctactgtttctctcttgccagggtattaggaaggacaggaaaagtaggaggaggatctggggcattgccctaggaaatgaaagaattgtgtatagaatggaagggggatcatcaaggacatgtatctcaaattttctttgagatgcaggttagttgaccttgctgcagttctccttcccattaattcattgggatggaagccaaaaataaaagaggtgcctctgaggattagggttgagcactcaagggaaagatggagtagagggcaaatagcaaaagttgttgcactcctgaaattctattaacatttccgcagaagatgagtagggagatgctgccttcccttttgagatagtgtagaaaaacactagatagtgtgagaggttcctttctgtccattgaaacaaggctaaggatactaccaactactatcaccatgaccattgtactgacaacaattgaatgcagtctccctgcagggcagattatgccaggcactttacatttgttgatcccatttgacattcacaccaaagctctgagttccattttacagctgaagaaattgaagcttagagaaattaagaagcttgtttaagtttacacagctagtaagagttttaaaaatctctgtgcagaagtgttggctgggtgctctccccaccactacccttgtaaacttccaggaagattggttgaaagtctgaataaaagctgtcctttcctaccaatttcctccccctcctcactctcacaagaaaaccaaaagtttctcttcagagttgttgactcatagtacagtaaagggtggaggtgatatggcattctgaaagtagggagggactaagtcagtcgtcatactaaacaaaaatcccagtaccctttccttatttagctaccagaatgatgacagacagactcaaaccctgtcaagacaggaccctggaggatcctcctatggggaaactgcataatctgaaaaaaaggctgacagatacaaatagttgaggatccttcaacacaaaagctgcgtgtctgccctattactcacctgacgatgaagcccacaatgcaacaaatcccttccactcacagcatttggtcaacctgtcagagcctcattcttaattatgaaaagtagtcaatgaacacaatacatttgagggaaaggccgtaacatgaaaggcaccaacaaataaatgtgagagtagatagataccttttgagtataaaagatcctccaaaataagagattaaaccaagatagaggaaaacacagtagctgggaaacaaggaatccaacgcaggagaaagtgaagagaattcccaggatgttggtgtgtagcaggcttagaaagc

SEQ ID NO: 2 - protein, 346 aa, Homo sapiens
MERKFMSLQPSISVSEMEPNGTFSNNNSRNCTIENFKREFFPIVYLIIFFWGVLGNGLSIYVFLQPYKKSTSVNVFMLNLAISDLLFISTLPFRADYYLRGSNWIFGDLACRIMSYSLYVNMYSSIYFLTVLSVVRFLAMVHPFRLLHVTSIRSAWILCGIIWILIMASSIMLLDSGSEQNGSVTSCLELNLYKIAKLQTMNYIALVVGCLLPFFTLSICYLLIIRVLLKVEVPESGLRVSHRKALTTIIITLIIFFLCFLPYHTLRTVHLTTWKVGLCKDRLHKALVITLALAAANACFNPLLYYFAGENFKDRLKSALRKGHPQKAKTKCVFPVSVWLRKETRV

SEQ ID NO: 3 - DNA, 32 bp, Artificial Sequence
  Synthetic Construct
actaagtcagtcatcatactaaacaaaaatcc

SEQ ID NO: 4 - DNA, 31 bp, Artificial Sequence
  Synthetic Construct
actaagtcagtcgtcatactaaacaaaaatc

SEQ ID NO: 5 - DNA, 21 bp, Artificial Sequence
  Synthetic Construct
gggtggaggtgatatggcatt

SEQ ID NO: 6 - DNA, 25 bp, Artificial Sequence
  Synthetic Construct
tctgtctgtcatcattctggtagct

SEQ ID NO: 7 - DNA, 27 bp, Artificial Sequence
  Synthetic Construct
ttgaggatccttcaacacaaaagctgc

SEQ ID NO: 8 - DNA, 26 bp, Artificial Sequence
  Synthetic Construct
ttgaggatccttcagcacaaaagctg

SEQ ID NO: 9 - DNA, 25 bp, Artificial Sequence
  Synthetic Construct
agacagactcaaaccctgtcaagac

SEQ ID NO: 10 - DNA, 25 bp, Artificial Sequence
  Synthetic Construct
gcttcatcgtcaggtgagtaatagg

SEQ ID NO: 11 - DNA, 30 bp, Artificial Sequence
  Synthetic Construct
tgctaagctgcagaccatgaactatattgc

SEQ ID NO: 12 - DNA, 29 bp, Artificial Sequence
  Synthetic Construct
ctaagctgcagaccgtgaactatattgcc

SEQ ID NO: 13 - DNA, 25 bp, Artificial Sequence
  Synthetic Construct
cacatcatgcttagagctgaatctc

SEQ ID NO: 14 - DNA, 26 bp, Artificial Sequence
  Synthetic Construct
cagataacagatgctgagtgtgaaaa